(19) United States
(12) Patent Application Publication
(10) Pub. No.: US 2009/0299478 A1
(43) Pub. Date: Dec. 3, 2009

(54) Lordotic Implant for Posterior Approach

(75) Inventors: Thomas Carls, Memphis, TN (US); Danny Braddock, Memphis, TN (US); Eric Lange, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/131,995

(22) Filed: Jun. 3, 2008

(51) Int. Cl. A61F 2/44

(52) U.S. Cl. 623/17.16

(57) ABSTRACT

An intervertebral implant for positioning in a lordotic disc space that avoids any unnecessary cutting of bone and therefore, any unwanted subsidence, is provided. Specifically, the implant can work particularly well between the L5-S1 juncture. The implant comprises an inferior component, and a superior component, wherein the superior component has an upper portion and a lower portion, wherein an expandable component is situated between the upper portion and the lower portion, and the implant has a profile that changes from a relatively flat profile to a relatively angled profile. A method for inserting an intervertebral implant into a lordotic intervertebral disc space from a posterior approach without unnecessary bone removal, and without resulting subsidence also is provided.

### TECHNICAL FIELD

The present invention relates generally to intervertebral implants and more particularly, to spinal implants for use between the fifth lumbar vertebra (L5) and the sacrum (S1).

### BACKGROUND

The human spine is a biomechanical structure with thirty-three vertebral members, and is responsible for protecting the spinal cord, nerve roots and internal organs of the thorax and abdomen. The spine also provides structural support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, be caused by the natural aging process, or may be the result of a degenerative disease or condition.

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

It is known that intervertebral discs are prone to injury and degeneration. For example, herniated discs are common, and typically occur when normal wear, or exceptional strain, causes a disc to rupture. Degenerative disc disease typically results from the normal aging process, in which the tissue gradually looses its natural water and elasticity, causing the degenerated disc to shrink and possibly to rupture. These conditions often are treated with the use of intervertebral implants. The region between the fifth lumbar vertebra and the sacrum juncture (or “L5-S1” region) is an area that is particularly prone to problems.

Thus, the L5-S1 region is an area that often is treated with implant techniques. The L5-S1 region, however, is such that the distance between the bony structures increases greatly in the posterior to anterior direction. This angle is referred to as the “lordotic angle”. Certain implants, including those that are put in place from an anterior direction, are able to match the so-called L5-S1 lordotic angle only through the use of implants that match the particular angled endplates. Furthermore, the posterior introduction of a suitable implant for the L5-S1 region is very challenging.

That is, when inserting an implant from a posterior approach, one often removes bone, including the relatively hard endplate material (typically from the superior vertebra), to form a more parallel approach angle for an implant. This, however, will result in subsidence, which is unwanted movement of the implant into the vertebra where the bone and endplate was removed.

There, therefore, is a need for a spinal implant in lordotic junctures that can be inserted from a posterior approach, but also does not lead to subsidence because of unnecessary bone removal.

### SUMMARY

An intervertebral implant for positioning in a lordotic disc space that avoids any unnecessary cutting of bone and therefore, any unwanted subsidence, is provided. Specifically, the implant can work particularly well between the L5-S1 juncture. The implant comprises an inferior component, and a superior component, wherein the superior component has an upper portion and a lower portion, wherein an expandable component is situated between the upper portion and the lower portion, and the implant has a profile that changes from a relatively flat profile to a relatively angled profile.

In certain embodiments, the upper portion and/or the lower portion of the intervertebral implant rotate about a common point. In some embodiments, the expandable component of the intervertebral implant comprises a balloon material. In some embodiments, the material that is used to expand the expandable component, i.e., the injection material is cement material, whereas in other embodiments, it is a polymer.

In certain embodiments of the invention, the intervertebral implant is a bilateral implant in the same intervertebral disc space. In some embodiments of the intervertebral implant, the implant is motion preserving, often wherein motion is preserved between the inferior and superior components. In other embodiments, the implant is a fusion device.

A method for inserting an intervertebral implant into a lordotic intervertebral disc space from a posterior approach without unnecessary bone removal, and without resulting subsidence also is provided. The method comprises the steps of (1) inserting a low profile intervertebral implant from the posterior approach, the implant comprising an expandable component; and (2) expanding the expandable component until the implant fits properly within the lordotic disc space. The method may further comprise the steps of using an injection tube to insert a material into the expandable component, and thereafter, removing the injection tube. In addition, the method may further comprise the steps of using a balloon as the expandable component and a cement as the material to expand the balloon.

Additional aspects and features of the present disclosure will be apparent from the detailed description and claims as set forth below.

### BRIEF DESCRIPTION OF THE DRAWINGS

FIG. 1 is a diagrammatic, side view of a cut-away section of a spinal column from the L4 to S1 vertebrae.

FIG. 2 is a side view of one embodiment of a spinal implant of the present invention.

FIG. 3A is an isometric rear view of the implant of FIG. 2 without the injection tube in place.

FIG. 3B is an isometric rear view of the implant of FIG. 2 with the injection tube in place.

FIG. 4 is a side view of the implant of FIG. 2 in an expanded state.

FIG. 5 is an exploded isometric view of the implant of FIG. 2 in a relatively expanded state.

FIG. 6A is a side view of the implant of FIG. 2 undergoing flexion.

FIG. 6B is a side view of the implant of FIG. 2 in a neutral position.

FIG. 6C is a side view of the implant of FIG. 2 undergoing extension.

FIG. 7 is a side view of the implant of FIG. 2 in cooperation with the L5-S1 juncture of the spinal column.

FIG. 8 is a front view of the implant of FIG. 2 of the present invention in a bilateral arrangement in cooperation with the L5-S1 juncture of the spinal column.

FIG. 9 is a side view of an implant of FIG. 2 in cooperation with the L5-S1 juncture of the spinal column, when the implant is in an expanded state.

FIG. 10A is a side view of an alternate embodiment of a spinal implant according to the present invention, in an un-expanded state.

FIG. 10B is a side view of the implant of FIG. 10A, without the inferior component, in an expanded state.

FIG. 11A is an alternate embodiment of a spinal implant according to the present invention, in an un-expanded state.

FIG. 11B shows the implant of FIG. 11A, without the inferior component, in an expanded state.

### DETAILED DESCRIPTION

For the purpose of promoting an understanding of the principles of the present disclosure, reference is made to the specific embodiments illustrated in the drawings, and specific language is used to describe the embodiments. It is nevertheless understood that no limitation of the scope of the present disclosure is intended. Any alterations and further modifications of the described embodiments, and any further applications of the principles of the present disclosure as described herein, are fully contemplated, as would occur to one skilled in the art to which the invention relates.

To avoid the unwanted subsidence, as well as any unnecessary cutting of bone, the present invention provides a spinal implant that can work with the lordotic angle found between some vertebrae segments, and particularly between the L5-S1 juncture. More particularly, the present invention provides a spinal implant that can be inserted posteriorly into the L5-S1 juncture, will not require cutting and therefore, not lead to subsidence in the future.

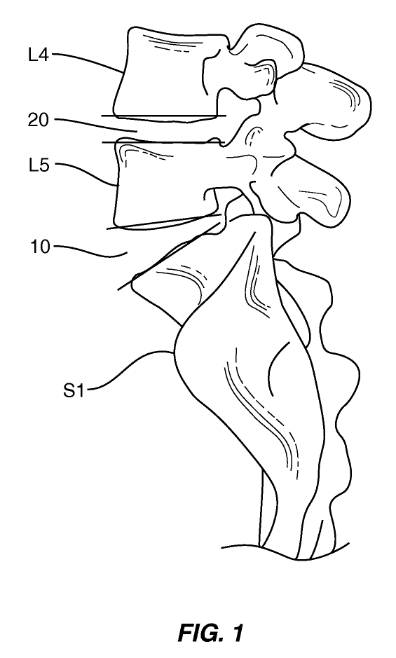

FIG. 1 shows a diagrammatic, side view of a cut-away section of a spinal column from the L4 to S1 vertebrae. In particular, FIG. 1 shows how the L5-S1 juncture 10 has a greater angle toward the anterior side of the spinal column, i.e., is lordotic, as compared to the L4-L5 juncture 20, which is relatively parallel. As described above, when inserting an implant from the posterior approach, because of the removal of bone, which includes the relatively hard endplate material, this often results in subsidence.

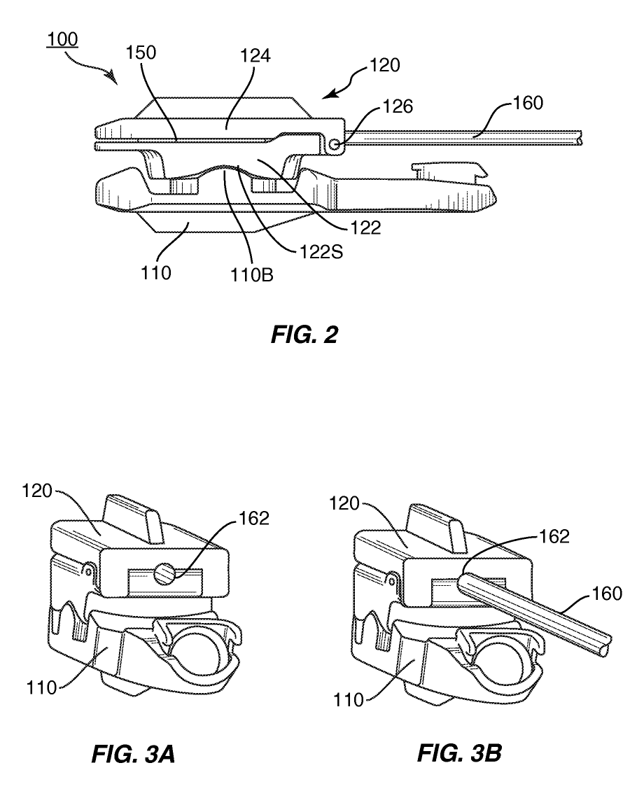

FIG. 2 shows a side view of one embodiment of a spinal implant 100 of the present invention. The spinal implant 100 allows for dynamics or motion after implantation between the L5 and S1 vertebrae. That is, the spinal implant 100 has a ball-and-socket type of mechanism, and other similarities to that described in commonly assigned and co-pending patent application, entitled “Posterior Total Joint Replacement,” having application Ser. No. 11/757,084, which was filed Jun. 20, 2007, and which is incorporated herein by reference in its entirety.

As shown in FIG. 2, spinal implant 100 has an inferior component 110, a superior component 120, and an expandable component 150. The superior component 120 includes a lower portion 122 and an upper portion 124. The inferior component 110 has a portion 110B that serves as a ball and the lower portion 122 of the superior component 120 has a portion 122S that serves as the socket in the ball-and-socket type of union of the two components 110 and 120 that form the implant 100. Note, however, as shown in FIG. 2, that the sides of ball portion 110B are “cut off” and accordingly, the ball portion 110B is not 360 degrees in circumference in implant 100.

FIG. 2 also shows an injection tube 160, which is protruding outside of the right side of the implant 100, and more specifically, the right side of the superior component 120. The injection tube 160 is provided for facilitating expansion of the expandable component 150. FIG. 3A shows an isometric rear view of the implant 100 without the injection tube 160 in place, depicting a hole 162 in the superior component 120 where the injection tube 160 is situated. FIG. 3A shows an isometric rear view of the implant 100 with the injection tube 160 in place through hole 162 in the superior component 120. In both FIG. 3A's, the implants 100 are shown in the un-expanded state.

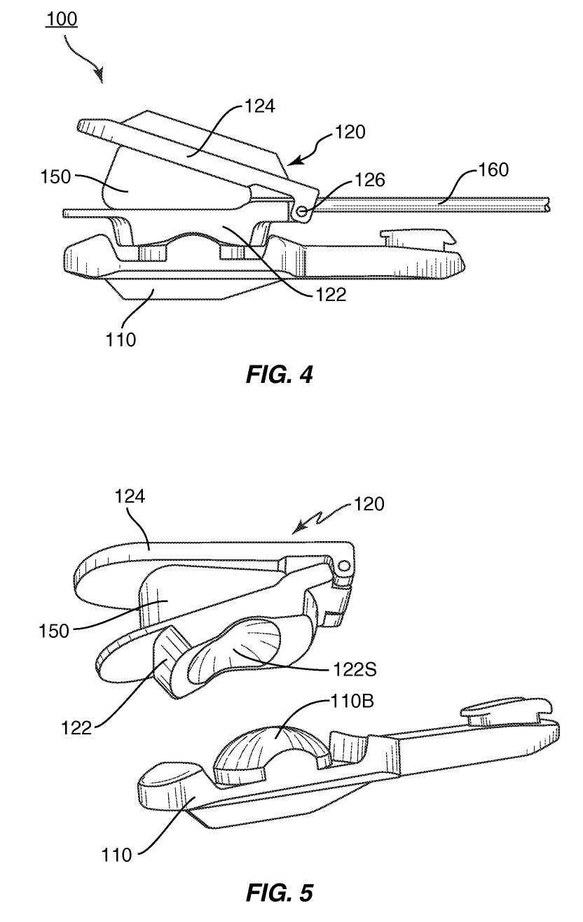

FIG. 4 shows the implant 100 in an expanded state. Specifically, when an injection material is inserted or injected through the injection tube 160, expandable component 150 expands and the upper portion 124 of the superior component 120 rotates (clockwise as shown in FIG. 4) around pivot point 126. Thus, while FIG. 2 shows the implant 100 in a relatively thin and flat, un-expanded state, FIG. 4 shows the implant 100 in a relatively thicker expanded state, i.e., because of its oblique nature.

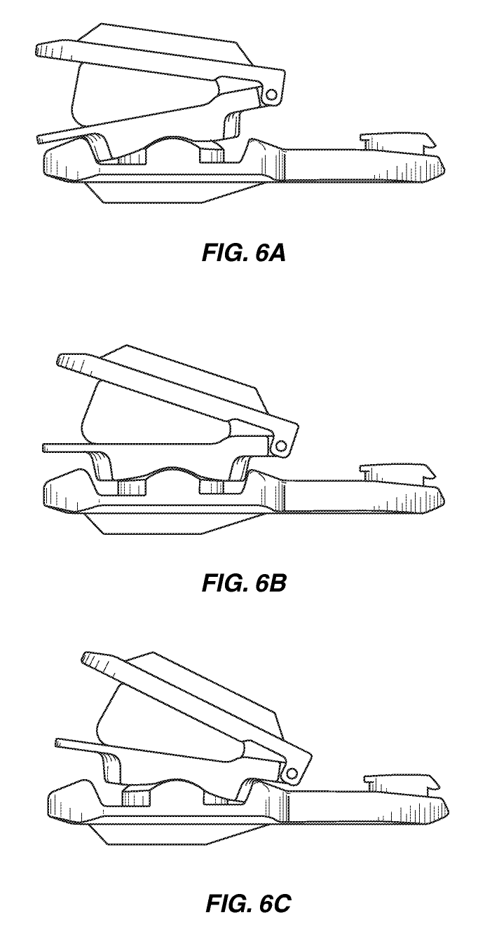

FIG. 5 shows an exploded isometric view of the implant 100 in a relatively expanded state, detailing the ball-and-socket juncture. Specifically, FIG. 5 shows the curvature of the ball portion 110B of the inferior component 110 and the socket portion 122S of the superior component 120. FIGS. 6A, 6B, and 6C show the range of motion the implant 100 can achieve when in an expanded state. Specifically, FIG. 6A shows the implant 100 undergoing flexion (with reference to the spine of FIG. 1, for example), FIG. 6B shows the implant 100 in a neutral position, while FIG. 6C shows the implant 100 undergoing extension.

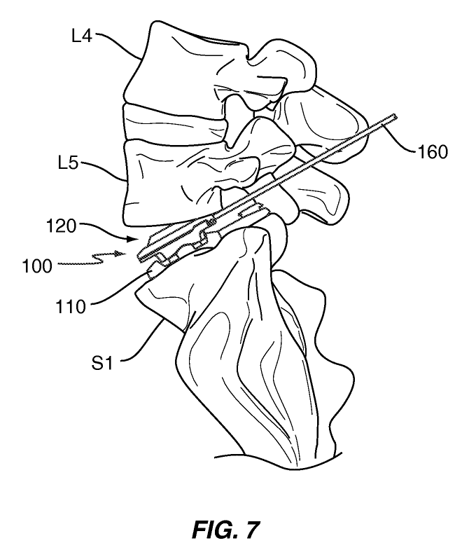

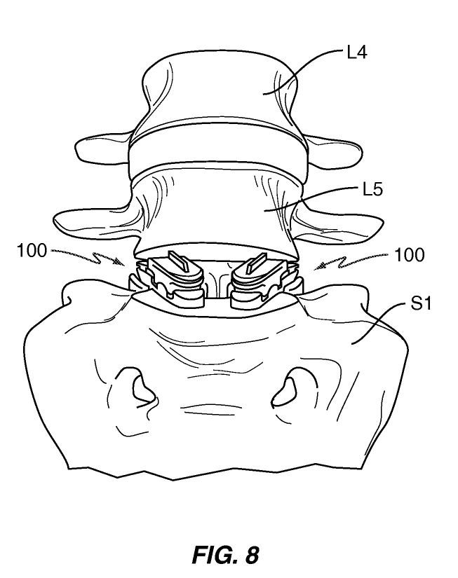

FIG. 7 shows a side view of the implant 100 in cooperation with the L5-S1 juncture of the spinal column. In particular, as shown, the implant 100 is inserted when it is still in its un-expanded state. As shown in FIG. 8, the implant 100 of the present invention is preferably utilized in a bilateral arrangement, i.e., spinal implants 100 and 100 are shown inserted side by side, but spaced apart in the same intervertebral disc space, although the implant 100 of the present invention is not limited to a bilateral arrangement. Expanding the implant after it is inserted allows for insertion of the implant 100 without the removal of any unnecessary bone or endplate material from the L5 or S1 vertebrae. Naturally, with the embodiment of implant 100, there may be some milling of the S1 endplate to facilitate placement and mating of the inferior component 110 on the endplate of the sacrum S1, or similarly, to facilitate mating of the upper portion 124 of the superior component 120 on the L5 vertebra after the implant is expanded.

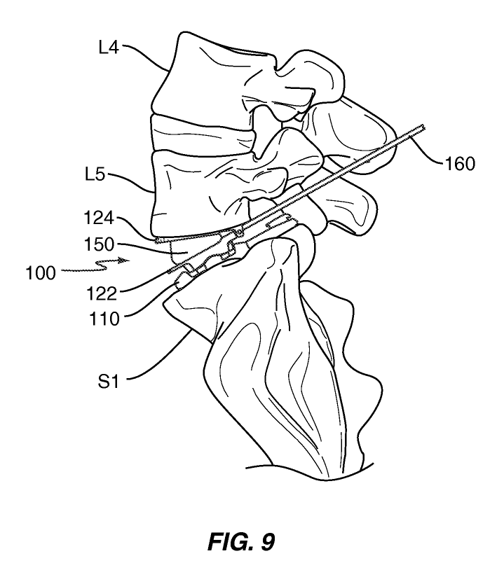

That is, after the implant is inserted as shown in FIG. 7, it is then expanded. FIG. 9 shows the implant 100 in cooperation with the L5-S1 juncture of the spinal column, when the implant 100 is in an expanded state. A material such as bone cement is injected through the injection tube 160 and received in the expandable component 150. After the expandable component 150 and implant 100 is expanded to the extent that the upper portion 124 of the superior component 120 mates with the L5 vertebra, the implant 100 has been expanded sufficiently and the injection tube 160 should be withdrawn. At this point, the expandable component 150 is either sealed, or alternatively, a one-way valve is used to expand the expandable component 150 to prevent material from escaping therefrom.

As shown in FIG. 9, the upper portion 124 of the superior component 120 is mated with the L5 vertebra, the inferior component 110 is mated with the sacrum S1, and the expandable component 150 has allowed the lordotic angle to be filled in a posterior approach without unnecessary bone removal. Also as shown in FIG. 9, the lower portion 122 of the superior component 120 and the inferior component 110 allows for the spinal implant 100 to preserve motion. Alternatively, within the spirit of the invention, i.e., if one wants to utilize the spinal implant as a fusion device, one merely needs to join the lower portion 122 of the superior component 120 and the inferior component 110 together so there will be no ball-and-socket type of mechanism, but one stationary component or a stationary union of two components.

In some embodiments, the expandable component 150 may be a balloon, or similar material, but biocompatible and strong enough to withstand the relatively harsh environment. Specifically, the expandable component 150 can be made from one or more elastic biocompatible materials. For example, the materials used to make up the expandable component 150 can be silicone, polyurethane, polycarbonate urethane, polyethylene terephthalate, silicone copolymers, polyolefin, or any combination thereof.

The particular material used to expand the expandable component 150, i.e., the injection material injected through the injection tube 160 and into the expandable component 150 can be a material extremely rigid such as a bone cement or can be a polymer with a lower modulus of elasticity that allows for some shock absorption. For example, the injection material may be a biocompatible material that becomes substantially rigid after curing, or a polymer material that becomes substantially rigid yet remains elastic after curing. Also, the injectable biocompatible material can include ceramics or fluids.

As for more specific examples, the injection material can be polymer materials that can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefm, and flouropolyolefin.

As for further specific examples, the expandable component 150 can be hydrogels, which can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

As for even further specific examples, the injection material can be ceramics, which can include calcium phosphate, hydroxyapatite, calcium sulfate, bioactive glass, or a combination thereof. Alternatively, the injection material can be one or more fluids such as sterile water, saline, or sterile air.

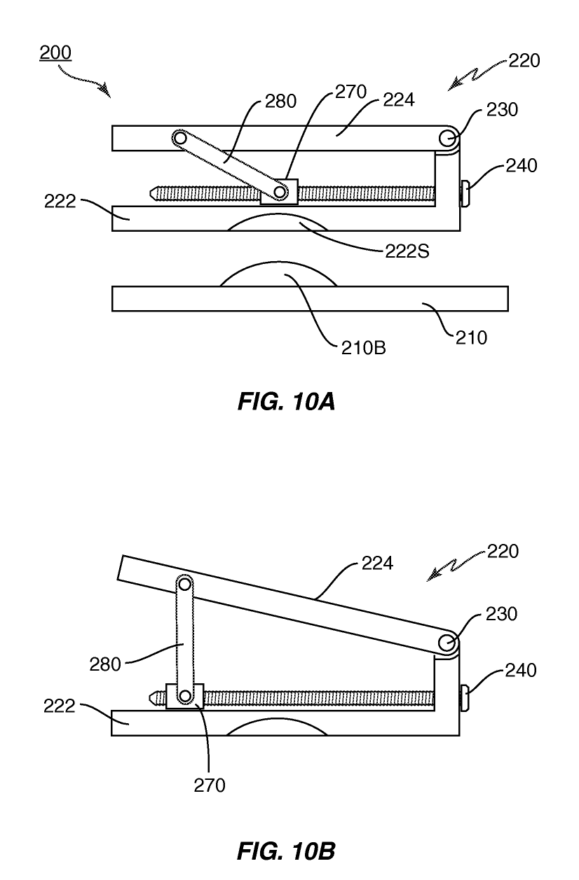

FIG. 10A shows an alternate embodiment of a spinal implant 200 according to the present invention, in an un-expanded state. Similar to implant 100, implant 200 can be inserted into the L5-S1 intervertebral disc space (or other lordotic juncture) from the posterior side of the spinal column without unnecessary bone removal. Implant 200 has an inferior component 210, a superior component 220, a screw 240, a slider 270, and an adjusting arm 280. As with superior component 120 of implant 100, the superior component 220 includes a lower portion 222 and an upper portion 224. As with implant 100, the inferior component 210 has a portion 210B that serves as a ball and the lower portion 222 of the superior component 220 has a portion 222S that serves as the socket in a ball-and-socket type of union of the two components 210 and 220 that form the implant 200. Thus, the spinal implant 100 allows for dynamics or motion after implantation between the L5 and S1 vertebrae, or other lordotic disc space. As with implant 100, however, one may utilize the spinal implant 200 as a fusion device by merely joining the lower portion 222 of the superior component 220 and the inferior component 210 together so there will be no ball-and-socket type of mechanism, but one stationary component or a stationary union of two components.

As mentioned above, FIG. 10A shows implant 200 in an un-expanded state. It is inserted into a lordotic disc space as shown, as it is relatively flat, and can be inserted from a posterior approach. After the implant 200 is in position in the disc space, with the inferior component 210 mated with its respective vertebral endplate, the upper portion 224 of the superior component 220 can then be moved into position against its respective vertebral endplate. As the screw 240 is rotated, the slider 270 situated on the screw 240 moves in the anterior direction, which thereby rotates the adjusting arm 280 in the clockwise direction, which thereby rotates the upper portion 224 of the superior component 220 in the clockwise direction as it rotates about pivot point 230. FIG. 10B shows implant 200 (without the inferior component) in an expanded state, with the slider 270 in a more anterior position, the adjusting arm in a more clockwise direction, and the upper portion 224 of the superior component 220 in a more clockwise direction, which results in a more angled, or more lordotic spinal implant 200.

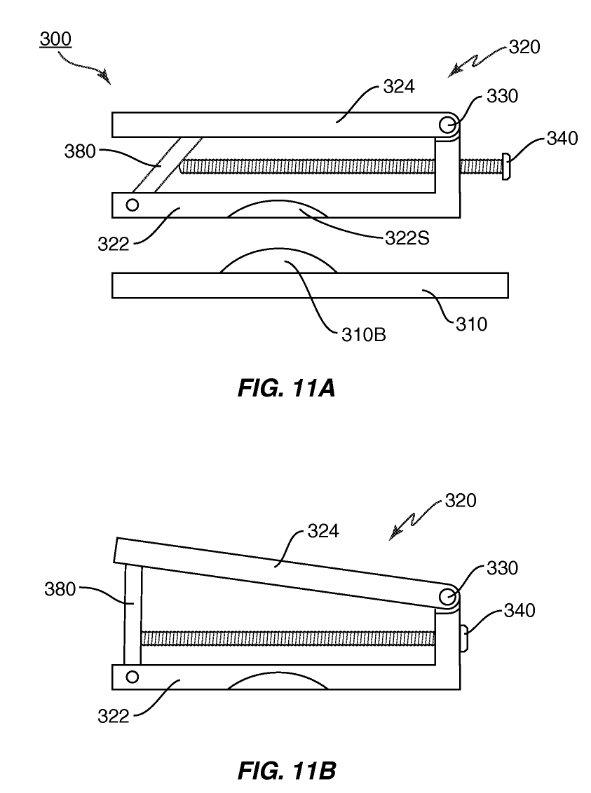

FIG. 11A shows an alternate embodiment of a spinal implant 300 according to the present invention, in an un-expanded state. Similar to implant 200, implant 300 can be inserted into the L5-S1 intervertebral disc space (or other lordotic juncture) from the posterior side of the spinal column without unnecessary bone removal. Implant 300 has an inferior component 310, a superior component 320, a screw 340, and an adjusting arm 380. As above, the superior component 320 includes a lower portion 322 and an upper portion 324. Also as above, the inferior component 310 has a portion 310B that serves as a ball and the lower portion 322 of the superior component 320 has a portion 322S that serves as the socket in a ball-and-socket type of union of the two components 310 and 320 that form the implant 300. Thus, the spinal implant 100 allows for dynamics or motion after implantation between the L5 and S1 vertebrae, or other lordotic disc space. Same as above, however, one may utilize the spinal implant 300 as a fusion device by merely joining the lower portion 322 of the superior component 320 and the inferior component 310 together so there will be no ball-and-socket type of mechanism, but one stationary component or a stationary union of two components.

As mentioned above, FIG. 11A shows implant 300 in an un-expanded state. It is inserted into a lordotic disc space as shown, as it is relatively flat, and can be inserted from a posterior approach. After the implant 300 is in position in the disc space, with the inferior component 310 mated with its respective vertebral endplate, the upper portion 324 of the superior component 320 can then be moved into position against its respective vertebral endplate. As the screw 340 is rotated, the screw 340 moves in the anterior direction and moves and rotates the adjusting arm 380 in the counter-clockwise direction, which thereby rotates the upper portion 324 of the superior component 320 in the clockwise direction as it rotates about pivot point 330. FIG. 11B shows implant 300 (without the inferior component) in an expanded state, with screw 340 in a more anterior position, the adjusting arm 380 in a more counter-clockwise direction (its distal end slides within the upper portion 324 of the superior component 320), and the upper portion 324 of the superior component 320 in a more clockwise direction, which results in a more angled, or more lordotic spinal implant 300.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed:

1. An intervertebral implant for positioning between an upper vertebra and a lower vertebra, the implant comprising:
an inferior component;
a superior component having an upper portion and a lower portion; and
an expandable component situated between the upper portion and the lower portion, wherein the implant has a profile that changes from a relatively flat profile to a relatively angled profile.

2. The intervertebral implant of claim 1, wherein the upper portion and/or the lower portion rotate about a common point.

3. The intervertebral implant of claim 1, wherein expandable component comprises a balloon material.

4. The intervertebral implant of claim 3, wherein the expandable component further comprises a cement material.

5. The intervertebral implant of claim 3, wherein the expandable component further comprises a polymer.

6. The intervertebral implant of claim 1, wherein the implant is a bilateral implant in the same intervertebral disc space.

7. The intervertebral implant of claim 1, wherein the implant is motion preserving.

8. The intervertebral implant of claim 7, wherein motion is preserved between the inferior and superior components.

9. The intervertebral implant of claim 1, wherein the implant is a fusion device.

10. An intervertebral implant for positioning between an upper vertebra and a lower vertebra, the implant comprising:
an inferior component;
a superior component having an upper portion and a lower portion, wherein the upper portion and/or the lower portion rotate about a common point; and
an expandable component situated between the upper portion and the lower portion, wherein the implant has a profile that changes from a relatively flat profile to a relatively angled profile.

11. The intervertebral implant of claim 10, wherein expandable component comprises a balloon material.

12. The intervertebral implant of claim 11, wherein the expandable component further comprises a cement material.

13. The intervertebral implant of claim 11, wherein the expandable component further comprises a polymer.

14. The intervertebral implant of claim 10, wherein the implant is a bilateral implant in the same intervertebral disc space.

15. The intervertebral implant of claim 10, wherein the implant is motion preserving.

16. The intervertebral implant of claim 15, wherein motion is preserved between the inferior and superior components.

17. The intervertebral implant of claim 10, wherein the implant is a fusion device.

18. A method for inserting an intervertebral implant into a lordotic intervertebral disc space from a posterior approach without unnecessary bone removal, and resulting subsidence, the method comprising the following steps:
inserting a low profile intervertebral implant from the posterior approach, the implant comprising an expandable component; and
expanding the expandable component until the implant fits properly within the lordotic disc space.

19. The method of inserting an intervertebral implant into a lordotic intervertebral disc space of claim 18, wherein the step of expanding further comprises using an injection tube to insert a material into the expandable component, and thereafter, removing the injection tube.

20. The method of inserting an intervertebral implant into a lordotic intervertebral disc space of claim 18, wherein the step of expanding further comprises using a balloon as the expandable component and a cement as the material to expand the balloon.
